(19) United States
(12) Patent Application Publication
(10) Pub. No.: US 2025/0143806 A1
(43) Pub. Date: May 8, 2025

(54) DETECTING AND DISTINGUISHING CRITICAL STRUCTURES IN SURGICAL PROCEDURES USING MACHINE LEARNING

(71) Applicant: DIGITAL SURGERY LIMITED, London (GB)

(72) Inventors: David P. Owen, London (GB); Maria Grammatikopoulou, London (GB); Imanol Luengo Muntion, Bilbao (ES); Danail V. Stoyanov, London (GB)

(21) Appl. No.: 18/727,739

(22) PCT Filed: Jan. 28, 2022
(86) PCT No.: PCT/GR2022/000004

§ 371 Date: Jul. 10, 2024

(51) Int. Cl. A61B 34/00; A61B 1/00; G06T 7/00; G06T 19/00; G06V 10/764; G06V 10/82; G06V 20/20; G06V 20/40; G06V 20/70

(52) U.S. Cl. CPC A61B 34/25; A61B 1/000094; A61B 1/000096; G06T 7/0012; G06T 19/006; G06V 10/764; G06V 10/82; G06V 20/20; G06V 20/41; G06V 20/70; G06T 2207/20084; G06T 2207/30101; G06T 2210/12; G06T 2210/41; G06T 2219/004; G06V 2201/034

(57) ABSTRACT

Technical solutions are provided to facilitate computer assistance during a surgery to prevent complications by detecting, identifying, and highlighting specific anatomical structures in a video of the surgery using machine learning. According to some aspects, a computer vision system is trained to detect several structures in the video of the surgery, and further to distinguish between the structures despite their similar appearance.

### CROSS-REFERENCE TO RELATED APPLICATION(S)

This application claims the benefit of U.S. Provisional Application No. 63/163,425, filed Mar. 19, 2021, and entitled “Detection of Critical Structures In Surgical Data Using Label Relaxation and Self-Supervision,” and U.S. Provisional Application No. 63/211,098, filed Jun. 16, 2021, and entitled “Prediction of Anatomical Structures In Surgical Data Using Machine Learning,” the content of which is incorporated herein by reference in its entirety.

### BACKGROUND

The present disclosure relates in general to computing technology and relates more particularly to computing technology for automatic detecting and distinguishing critical structures in surgical procedures using machine learning, and providing user feedback based on the automatic detection.

Computer-assisted systems can be useful to augment a person's physical sensing, perception, and reaction capabilities. For example, such systems can effectively provide the information corresponding to an expanded field of vision, both temporal and spatial, that enables a person to adjust current and future actions and decisions based on the part of an environment not included in his or her physical field of view. Additionally, the systems can bring attention to occluded parts of the view, for example, due to structures, blood, etc. However, providing such information relies upon an ability to process part of this extended field in a useful manner. Highly variable, dynamic, and/or unpredictable environments present challenges in defining rules that indicate how representations of the environments are to be processed to output data to productively assist the person in action performance.

### SUMMARY

Technical solutions described herein include a computer-implemented method including detecting, using a first configuration of a neural network, a plurality of structures in a video of a laparoscopic surgical procedure. The method further includes identifying, using a second configuration of the neural network, from the plurality of structures, a first type of anatomical structure and a second type of anatomical structure. The method further includes generating an augmented video, the generating comprising annotating the video with the first type of anatomical structure and the second type of anatomical structure.

In one or more aspects, the surgical procedure is a laparoscopic cholecystectomy, the first type of anatomical structure is a cystic artery, and the second type of anatomical structure is a cystic duct.

In one or more aspects, an anatomical structure, from the plurality of structures, occludes at least one other anatomical structure from the plurality of structures in a frame of the video. In one or more aspects, the second configuration includes using one or more temporal models to provide context to the frame.

In one or more aspects, the neural network is trained to generate the second configuration based on weak labels.

In one or more aspects, the video is a live video stream of the surgical procedure.

In one or more aspects, the first type of anatomical structure is annotated differently than the second type of anatomical structure.

In one or more aspects, the annotating comprises adding, to the video, at least one from a mask, a bounding box, and a label.

Technical solutions described herein include a system that includes a training system configured to use a training dataset to train one or more machine learning models. The system further includes a data collection system configured to capture a video of a surgical procedure being performed. The system further includes a machine learning model execution system configured to execute the one or more machine learning models to perform a method. The method includes detecting a plurality of structures in the video by using a first configuration of the one or more machine learning models. The method further includes identifying, from the plurality of structures, at least one type of anatomical structure by using a second configuration of the one or more machine learning models. The system further includes an output generator configured to generate an augmented video by annotating the video to mark the at least one type of anatomical structure.

In one or more aspects, a first machine learning model is trained to detect the plurality of structures and a second machine learning model is trained to identify the at least one type of anatomical structure from the plurality of structures.

In one or more aspects, a same machine learning model is used to detect the plurality of structures and to identify the at least one type of anatomical structure from the plurality of structures. In one or more aspects, the same machine learning model, to detect the plurality of structures, uses the first configuration, which comprises a first set of hyperparameter values, and to identify the at least one type of anatomical structure, uses the second configuration, which comprises a second set of hyperparameter values.

In one or more aspects, the training system is further configured to train a third machine learning model to identify at least one surgical instrument from the plurality of structures.

In some aspects of the technical solutions, a computer program product includes a memory device having computer executable instructions stored thereon, which when executed by one or more processors cause the one or more processors to perform a method for prediction of features in surgical data using machine learning. The method includes detecting, using a neural network model, a plurality of structures in an input window comprising one or more images from a video of a surgical procedure, the neural network model is trained using surgical training data. The method further includes identifying, using the neural network model, at least one type of anatomical structure in the plurality of structures detected. The method further includes generating a visualization of the surgical procedure by displaying a graphical overlay at a location of the at least one type of anatomical structure in the video of the surgical procedure.

In one or more aspects, the neural network model detects the location of the at least one type of anatomical structure based on an identification of a phase of the surgical procedure being performed.

In one or more aspects, one or more visual attributes of the graphical overlay are configured based on the at least one type of anatomical structure. In one or more aspects, the one or more visual attributes assigned to the at least one type of anatomical structure are user configurable.

In one or more aspects, the neural network model is configured with a first set of hyperparameters to detect the plurality of structures, and with a second set of hyperparameters to identify the at least one type of anatomical structure.

In one or more aspects, the neural network model comprises a first neural network for semantic image segmentation and a second neural network for encoding.

In one or more aspects, the plurality of structures comprises one or more anatomical structures and one or more surgical instruments.

Additional technical features and benefits are realized through the techniques of the present invention. Aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the aspects of the invention are apparent from the following detailed description taken in conjunction with the accompanying drawings in which:

FIG. 1 shows an example snapshot of a laparoscopic cholecystectomy being performed;

FIG. 2 shows a system for detecting structures in surgical data using machine learning according to one or more aspects;

FIG. 3 depicts a flowchart of a method for detecting structures and distinguishing anatomical structures from the structural data in surgical data using machine learning according to one or more aspects;

FIG. 4 depicts a visualization of surgical data being used for training a machine learning model according to one or more aspects;

FIG. 5 depicts a second machine learning model used to detect structures in the surgical data according to one or more aspects;

FIG. 6 depicts example augmented visualizations of surgical views generated according to one or more aspects;

FIG. 7 depicts flow diagrams of depth estimation to retrieve a proxy for relative depth in the image;

FIG. 8 depicts a flow diagram of automatic prediction of anatomical structures in surgical data using machine learning according to one or more aspects;

FIG. 9 depicts a computer system in accordance with one or more aspects; and

FIG. 10 depicts a surgical procedure system in accordance with one or more aspects.

The diagrams depicted herein are illustrative. There can be many variations to the diagram or the operations described therein without departing from the spirit of the invention. For instance, the actions can be performed in a differing order, or actions can be added, deleted, or modified. Also, the term “coupled” and variations thereof describe having a communications path between two elements and do not imply a direct connection between the elements with no intervening elements/connections between them. All of these variations are considered a part of the specification.

### DETAILED DESCRIPTION

Exemplary aspects of technical solutions described herein relate to, among other things, devices, systems, methods, computer-readable media, techniques, and methodologies for using machine learning and computer vision to improve surgical safety and workflow by automatically detecting one or more anatomical structures in surgical data, the structures being deemed to be critical for an actor involved in performing one or more actions during a surgical procedure (e.g., by a surgeon). In one or more aspects, the structures are detected dynamically and substantially in real-time as the surgical data is being captured by technical solutions described herein. A detected structure can be an anatomical structure, a surgical instrument, etc. Further, aspects of the technical solutions described herein address the technical challenge of distinguishing between structures where occluded views and/or lack of context challenge identification.

Description of the technical solutions herein is provided using laparoscopic cholecystectomy as an example surgical procedure. However it should be appreciated that the technical solutions described herein are not limited to only that type of surgical procedure. The technical solutions described herein are applicable to any other type of surgical procedure where detection of anatomical structures and distinguishing between the detected anatomical structures in a captured frame (e.g., image or video frame) is helpful.

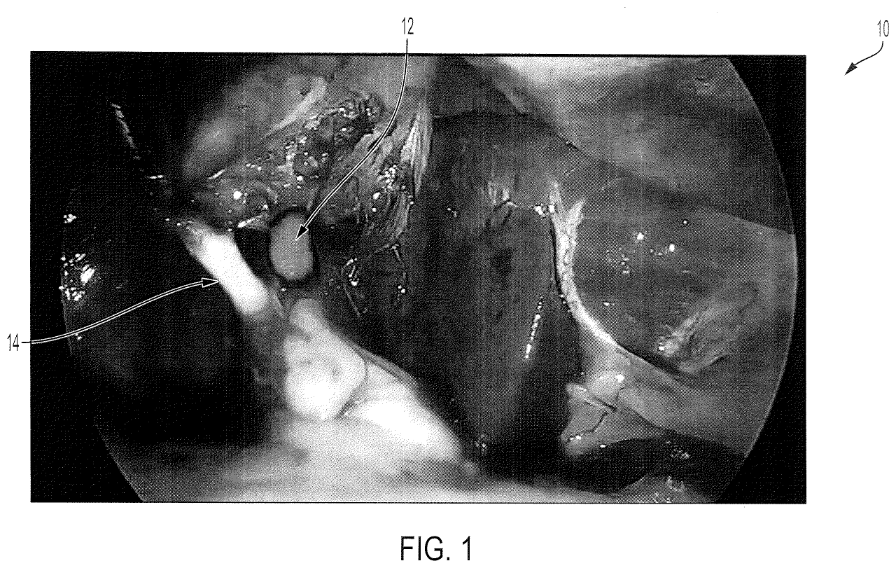

Laparoscopic cholecystectomy is a common surgery in which the gallbladder is removed. This involves exposing the cystic duct and cystic artery, clipping and dividing them, and then extracting the gallbladder. FIG. 1 shows an example snapshot 10 of a laparoscopic cholecystectomy with two anatomical structures labeled. In snapshot 10 shown in FIG. 1, the cystic artery 12 and cystic duct 14 are labeled. As can be seen, the two anatomical structures can be difficult to distinguish from each other simply by visual cues without context, such as the direction of viewing, location of the gall bladder, etc. Complications can occur when the structures are misidentified or confused with other structures in the vicinity, such as the common bile duct, particularly as they may be difficult to distinguish without thorough dissection.

Presently existing solutions provide official guidance that requires surgeons to establish a “critical view of safety” (CVS) before clipping and division. In CVS, both structures can clearly and separately be identified and traced as they enter the gallbladder. Some existing techniques create a bounding box detection system based on anatomical landmarks that include the common bile duct and the cystic duct 14 but not the cystic artery 12. Some existing techniques have used joint segmentation of the hepatobiliary anatomy and classification of CVS.

Technical solutions described herein use machine learning models with two different settings: first, a single “combined critical structures” settings to detect the structures; and second, separate “cystic artery” and “cystic duct” settings to classify the detected structures into the two respective types. In one or more aspects, the same machine learning model is used with different settings. In some aspects, different machine learning models are used in sequence, i.e., a first machine learning model with the first settings, and a second machine learning model with the second settings. As noted earlier, in other types of surgical procedures, where different types of structures are to be distinguished, different settings are used by the machine learning model.

In some instances, a computer-assisted surgical (CAS) system is provided that uses one or more machine learning models, trained with surgical data, to augment environmental data directly sensed by an actor involved in performing one or more actions during a surgical procedure (e.g., a surgeon). Such augmentation of perception and action can increase action precision, optimize ergonomics, improve action efficacy, enhance patient safety, and improve the standard of the surgical process.

The surgical data provided to train the machine learning models can include data captured during a surgical procedure, as well as simulated data. The surgical data can include time-varying image data (e.g., a simulated/real video stream from different types of cameras) corresponding to a surgical environment. The surgical data can also include other types of data streams, such as audio, radio frequency identifier (RFID), text, robotic sensors, other signals, etc. The machine learning models are trained to detect and identify, in the surgical data, “structures,” including particular tools, anatomical objects, actions being performed in the simulated/real surgical stages. In one or more aspects, the machine learning models are trained to define one or more parameters of the models so as to learn how to transform new input data (that the models are not trained on) to identify one or more structures. During the training, the models are input one or more data streams that may be augmented with data indicating the structures in the data streams, such as indicated by metadata and/or image-segmentation data associated with the input data. The data used during training can also include temporal sequences of one or more input data.

In one or more aspects, the simulated data can be generated to include image data (e.g., which can include time-series image data or video data and can be generated in any wavelength of sensitivity) that is associated with variable perspectives, camera poses, lighting (e.g., intensity, hue, etc.) and/or motion of imaged objects (e.g., tools). In some instances, multiple data sets can be generated—each of which corresponds to the same imaged virtual scene but varies with respect to perspective, camera pose, lighting, and/or motion of imaged objects, or varies with respect to the modality used for sensing, e.g., red-green-blue (RGB) images or depth or temperature. In some instances, each of the multiple data sets corresponds to a different imaged virtual scene and further varies with respect to perspective, camera pose, lighting, and/or motion of imaged objects.

The machine learning models can include a fully convolutional network adaptation (FCN) and/or conditional generative adversarial network model configured with one or more hyperparameters to perform image segmentation into classes. For example, the machine learning models (e.g., the fully convolutional network adaptation) can be configured to perform supervised, self-supervised, or semi-supervised semantic segmentation in multiple classes—each of which corresponding to a particular surgical instrument, anatomical body part (e.g., generally or in a particular state), and/or environment. Alternatively, or in addition, the machine learning model (e.g., the conditional generative adversarial network model) can be configured to perform unsupervised domain adaptation to translate simulated images to semantic segmentations. In one or more aspects, the machine learning model uses a neural network architecture of DeepLabV3+ and ResNet101 encoder. It is understood that other types of machine learning models or combinations thereof can be used in one or more aspects.

The trained machine learning model can then be used in real-time to process one or more data streams (e.g., video streams, audio streams, RFID data, etc.). The processing can include detecting and characterizing one or more structures within various instantaneous or block time periods. The structure(s) can then be used to identify the presence, position, and/or use of one or more features. Alternatively, or in addition, the structures can be used to identify a stage within a workflow (e.g., as represented via a surgical data structure), predict a future stage within a workflow, etc.

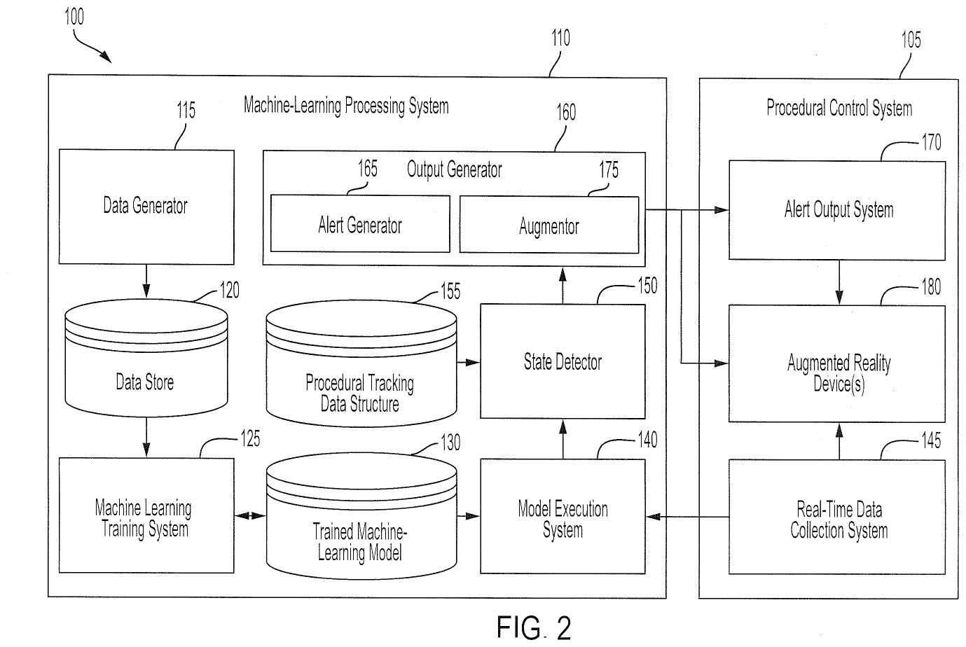

FIG. 2 shows a system 100 for detecting structures in surgical data using machine learning according to one or more aspects. System 100 uses data streams in the surgical data to identify procedural states according to some aspects. System 100 includes a procedural control system 105 that collects image data and coordinates outputs responsive to detected structures and states. The procedural control system 105 can include one or more devices (e.g., one or more user devices and/or servers) located within and/or associated with a surgical operating room and/or control center. System 100 further includes a machine learning processing system 110 that processes the surgical data using a machine learning model to identify a procedural state (also referred to as a phase or a stage), which is used to identify a corresponding output. It will be appreciated that machine learning processing system 110 can include one or more devices (e.g., one or more servers), each of which can be configured to include part or all of one or more of the depicted components of the machine learning processing system 110. In some instances, a part, or all of machine learning processing system 110 is in the cloud and/or remote from an operating room and/or physical location corresponding to a part, or all of procedural control system 105. For example, the machine learning training system 125 can be a separate device (e.g., server) that stores its output as the one or more trained machine learning models 130, which are accessible by the model execution system 140, separate from the machine learning training system 125. In other words, in some aspects, devices that “train” the models are separate from devices that “infer,” i.e., perform real-time processing of surgical data using the trained models 130.

Machine learning processing system 110 includes a data generator 115 configured to generate simulated surgical data, such as a set of virtual images, or record surgical data from ongoing procedures, to train a machine learning model. Data generator 115 can access (read/write) a data store 120 with recorded data, including multiple images and/or multiple videos. The images and/or videos can include images and/or videos collected during one or more procedures (e.g., one or more surgical procedures). For example, the images and/or video may have been collected by a user device worn by a participant (e.g., surgeon, surgical nurse, anesthesiologist, etc.) during the surgery, and/or by a non-wearable imaging device located within an operating room.

Each of the images and/or videos included in the recorded data can be defined as a base image and can be associated with other data that characterizes an associated procedure and/or rendering specifications. For example, the other data can identify a type of procedure, a location of a procedure, one or more people involved in performing the procedure, and/or an outcome of the procedure. Alternatively, or in addition, the other data can indicate a stage of the procedure with which the image or video corresponds, rendering specification with which the image or video corresponds and/or a type of imaging device that captured the image or video (e.g., and/or, if the device is a wearable device, a role of a particular person wearing the device, etc.). Further, the other data can include image-segmentation data that identifies and/or characterizes one or more objects (e.g., tools, anatomical objects, etc.) that are depicted in the image or video. The characterization can indicate the position, orientation, or pose of the object in the image. For example, the characterization can indicate a set of pixels that correspond to the object and/or a state of the object resulting from a past or current user handling.

Data generator 115 identifies one or more sets of rendering specifications for the set of virtual images. An identification is made as to which rendering specifications are to be specifically fixed and/or varied. Alternatively, or in addition, the rendering specifications that are to be fixed (or varied) are predefined. The identification can be made based on, for example, input from a client device, a distribution of one or more rendering specifications across the base images and/or videos, and/or a distribution of one or more rendering specifications across other image data. For example, if a particular specification is substantially constant across a sizable data set, the data generator 115 defines a fixed corresponding value for the specification. As another example, if rendering-specification values from at least a predetermined amount of data span across a range, the data generator 115 define the rendering specifications based on the range (e.g., to span the range or to span another range that is mathematically related to the range of distribution of the values).

A set of rendering specifications can be defined to include discrete or continuous (finely quantized) values. A set of rendering specifications can be defined by a distribution, such that specific values are to be selected by sampling from the distribution using random or biased processes.

One or more sets of rendering specifications can be defined independently or in a relational manner. For example, if the data generator 115 identifies five values for a first rendering specification and four values for a second rendering specification, the one or more sets of rendering specifications can be defined to include twenty combinations of the rendering specifications or fewer (e.g., if one of the second rendering specifications is only to be used in combination with an incomplete subset of the first rendering specification values or the converse). In some instances, different rendering specifications can be identified for different procedural phases and/or other metadata parameters (e.g., procedural types, procedural locations, etc.).

Using the rendering specifications and base image data, the data generator 115 generates simulated surgical data (e.g., a set of virtual images), which is stored at the data store 120. For example, a three-dimensional model of an environment and/or one or more objects can be generated using the base image data. Virtual image data can be generated using the model to determine—given a set of particular rendering specifications (e.g., background lighting intensity, perspective, zoom, etc.) and other procedure-associated metadata (e.g., a type of procedure, a procedural state, a type of imaging device, etc.). The generation can include, for example, performing one or more transformations, translations, and/or zoom operations. The generation can further include adjusting the overall intensity of pixel values and/or transforming RGB values to achieve particular color-specific specifications.

A machine learning training system 125 uses the recorded data in the data store 120, which can include the simulated surgical data (e.g., set of virtual images) and actual surgical data to train one or more machine learning models. The machine learning models can be defined based on a type of model and a set of hyperparameters (e.g., defined based on input from a client device). The machine learning models can be configured based on a set of parameters that can be dynamically defined based on (e.g., continuous or repeated) training (i.e., learning, parameter tuning). Machine learning training system 125 can use one or more optimization algorithms to define the set of parameters to minimize or maximize one or more loss functions. The set of (learned) parameters can be stored at a trained machine learning model data structure 130, which can also include one or more non-learnable variables (e.g., hyperparameters and/or model definitions).

A model execution system 140 can access the machine learning model data structure 130 and accordingly configure a machine learning model for inference (i.e., detection). The machine learning model can include, for example, a fully convolutional network adaptation, an adversarial network model, or other types of models as indicated in data structure 130. The machine learning model can be configured in accordance with one or more hyperparameters and the set of learned parameters.

The machine learning model, during execution, receives, as input, surgical data to be processed and generate an inference according to the training. For example, the surgical data can include data streams (e.g., an array of intensity, depth, and/or RGB values) for a single image or for each of a set of frames representing a temporal window of fixed or variable length in a video. The surgical data that is input can be received from a real-time data collection system 145, which can include one or more devices located within an operating room and/or streaming live imaging data collected during the performance of a procedure. The surgical data can include additional data streams such as audio data, RFID data, textual data, measurements from one or more instruments/sensors, etc., that can represent stimuli/procedural states from the operating room. The different inputs from different devices/sensors are synchronized before inputting in the model.

The machine learning model analyzes the surgical data, and in one or more aspects, detects and/or characterizes structures included in the visual data from the surgical data. The visual data can include image and/or video data in the surgical data. The detection and/or characterization of the structures can include segmenting the visual data or detecting the localization of the structures with a probabilistic heatmap. In some instances, the machine learning model includes or is associated with a preprocessing or augmentation (e.g., intensity normalization, resizing, cropping, etc.) that is performed prior to segmenting the visual data. An output of the machine learning model can include image-segmentation or probabilistic heatmap data that indicates which (if any) of a defined set of structures are detected within the visual data, a location and/or position, and/or pose of the structure(s) within the image data, and/or state of the structure(s). The location can be a set of coordinates in the image data. For example, the coordinates can provide a bounding box. Alternatively, the coordinates provide boundaries that surround the structure(s) being detected.

A state detector 150 can use the output from the execution of the machine learning model to identify a state within a surgical procedure (“procedure”). A procedural tracking data structure can identify a set of potential states that can correspond to part of a performance of a specific type of procedure. Different procedural data structures (e.g., and different machine learning-model parameters and/or hyperparameters) may be associated with different types of procedures. The data structure can include a set of nodes, with each node corresponding to a potential state. The data structure can include directional connections between nodes that indicate (via the direction) an expected order during which the states will be encountered throughout an iteration of the procedure. The data structure may include one or more branching nodes that feed to multiple next nodes and/or can include one or more points of divergence and/or convergence between the nodes. In some instances, a procedural state indicates a surgical action that is being performed or has been performed and/or indicates a combination of actions that have been performed. A “surgical action” can include an operation such as an incision, a compression, a stapling, a clipping, a suturing, a cauterization, a sealing, or any other such actions performed to complete a step/phase in the surgical procedure. In some instances, a procedural state relates to a biological state of a patient undergoing a surgical procedure. For example, the biological state can indicate a complication (e.g., blood clots, clogged arteries/veins, etc.), precondition (e.g., lesions, polyps, etc.).

Each node within the data structure can identify one or more characteristics of the state. The characteristics can include visual characteristics. In some instances, the node identifies one or more tools that are typically in use or availed for use (e.g., on a tool try) during the state, one or more roles of people who are typically performing a surgical task, a typical type of movement (e.g., of a hand or tool), etc. Thus, state detector 150 can use the segmented data generated by model execution system 140 that indicates the presence and/or characteristics of particular objects within a field of view to identify an estimated node to which the real image data corresponds. Identification of the node (and/or state) can further be based upon previously detected states for a given procedural iteration and/or other detected input (e.g., verbal audio data that includes person-to-person requests or comments, explicit identifications of a current or past state, information requests, etc.).

An output generator 160 can use the state to generate an output. Output generator 160 can include an alert generator 165 that generates and/or retrieves information associated with the state and/or potential next events. For example, the information can include details as to warnings and/or advice corresponding to current or anticipated procedural actions. The information can further include one or more events for which to monitor. The information can identify the next recommended action.

The user feedback can be transmitted to an alert output system 170, which can cause the user feedback to be output via a user device and/or other devices that is (for example) located within the operating room or control center. The user feedback can include a visual, audio, tactile, or haptic output that is indicative of the information. The user feedback can facilitate alerting an operator, for example, a surgeon or any other user of the system.

Output generator 160 can also include an augmentor 175 that generates or retrieves one or more graphics and/or text to be visually presented on (e.g., overlaid on) or near (e.g., presented underneath or adjacent to or on a separate screen) real-time capture of a procedure. Augmentor 175 can further identify where the graphics and/or text are to be presented (e.g., within a specified size of a display). In some instances, a defined part of a field of view is designated as being a display portion to include augmented data. In some instances, the position of the graphics and/or text is defined so as not to obscure the view of an important part of an environment for the surgery and/or to overlay particular graphics (e.g., of a tool) with the corresponding real-world representation.

Augmentor 175 can send the graphics and/or text and/or any positioning information to an augmented reality device 180, which can integrate the graphics and/or text with a user's environment in real-time as an augmented visualization. Augmented reality device 180 can include a pair of goggles that can be worn by a person participating in part of the procedure. It will be appreciated that, in some instances, the augmented display can be presented at a non-wearable user device, such as at a computer or tablet. The augmented reality device 180 can present the graphics and/or text at a position as identified by augmentor 175 and/or at a predefined position. Thus, a user can maintain a real-time view of procedural operations and further view pertinent state-related information.

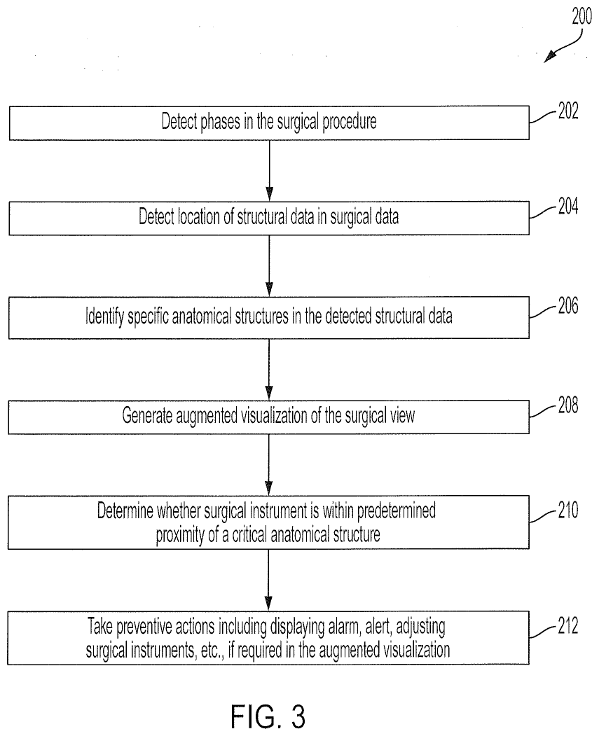

FIG. 3 depicts a flowchart of a method 200 for detecting and distinguishing anatomical structures in surgical data using machine learning according to one or more aspects. The method 200 can be executed by the system 100 as a computer-implemented method.

The method 200 includes training and using (inference phase) a first machine learning model 350 to detect surgical phases being performed in the procedure captured by the surgical data, at block 202. The phases can be determined using “operative workflow analysis,” which includes systematically deconstructing operations into steps and phases using machine learning. A “step” refers to the completion of a named surgical objective (e.g., hemostasis), while a “phase” represents a surgical event that is composed of a series of steps (e.g., closure). During each step, certain surgical instruments (e.g., forceps) are used to achieve a specific objective, and there is the potential for technical error (lapses in operative technique). Machine learning based recognition of these elements allows surgical workflow analysis to be generated automatically. Artificial deep neural networks (DNN), or other types of machine learning models, can be used to achieve automatic, accurate phase recognition in surgical procedures, such as cataract surgery, laparoscopic cholecystectomy, endoscopic endonasal transsphenoidal approach (eTSA) to resection of pituitary adenomas, or any other surgical procedure.

The machine learning model for detecting the phases includes a feature encoder to detect features from the surgical data for the procedure. The feature encoder can be based on one or more artificial neural networks, such as a convolutional neural network (CNN), a recurrent neural network (RNN), a feature pyramid network (FPN), a transformer network, or any other type of neural network or a combination thereof. The feature encoder can use a known technique, supervised, self-supervised, or unsupervised (e.g., autoencoder), to learn efficient data “codings” in the surgical data. The “coding” maps an input data to a feature space, which can be used by feature decoders to perform semantic analysis of the surgical data. In one or more aspects, the machine learning model includes task-specific decoders that detect instruments being used at an instance in the surgical data based on the detected features.

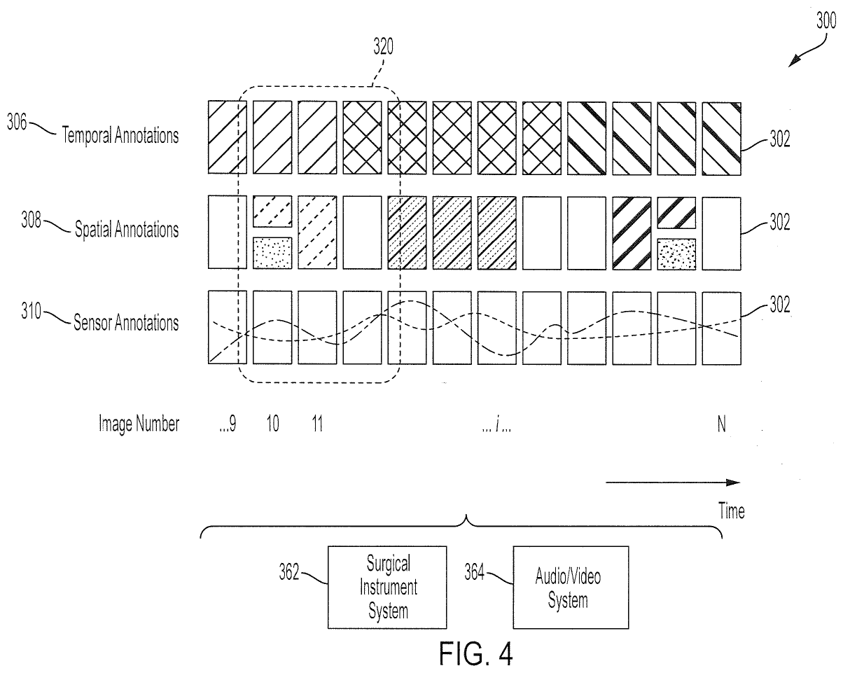

It should be noted that the machine learning model operates on the surgical data per frame, but can use information from a previous frame or a window of previous frames. FIG. 4 depicts a visualization of surgical data 300 being used for training a machine learning model according to one or more aspects. The depicted example surgical data 300 includes video data, i.e., a set of N images 302. The N images may be sequential in some examples. Alternatively, or in addition, the N images can be an arbitrarily sampled temporal set of frames/sensor/data information. The audiovisual data can be captured using an audio/video system 364. The audio/video system 364 can include one or more video capture devices that can include cameras placed in the surgical room to capture events surrounding (i.e., outside) the patient. In addition, or alternatively, the audio/video system 364 can include cameras that are passed inside (e.g., endoscopic cameras) the patient to capture endoscopic data. The endoscopic data provides video, images of the surgical procedure that are used to identify structures, such as anatomical structures, surgical instruments. For training the machine learning model, images 302, and other inputs are annotated. The annotations can include temporal annotations 306 that identify a surgical phase to which an image belongs or tracking information for different structures. Accordingly, a particular set or subset of sequential synchronized images from surgical data 302 (“images 302”) represents a surgical phase, or tracking state. The subset of sequential images 302 can include one or more images.

Further, the annotations can include spatial annotations 308 that identify one or more objects in the images 302. For example, the spatial annotations 308 can specify one or more regions of an image and identify respective objects in the regions. Further, an image 302 can include sensor annotations 310 that include values of one or more sensor measurements at the time the image 302 was captured. The sensor measurements can be from sensors associated with the patient, such as oxygen level, blood pressure, heart rate, etc. Alternatively, or in addition, the sensor measurements can be associated with one or more components being used in the surgical procedure, such as a brightness level of an endoscope, a fluid level in a tank, energy output from a generator, etc. Sensor measures can also come from real-time robotic systems indicating surgical activations or position or pose information about instruments. Other types of annotations can be used to train the machine learning model in other aspects.

In one or more examples, the sensor information can be received from a surgical instrument system 362. The surgical instrument system 362 can include electrical energy sensors, electrical impedance sensors, force sensors, bubble, occlusion sensors, and/or various other types of sensors. The electrical energy sensors can measure and indicate an amount of electrical energy applied to one or more surgical instruments being used for the surgical procedure. The impedance sensors can indicate an amount of impedance measured by the surgical instruments, for example, from the tissue being operated upon. The force sensors can indicate an amount of force being applied by the surgical instruments. Measurements from various other sensors, such as position sensors, pressure sensors, flow meters, can also be input. Such instrument data can be used to train machine learning algorithms to determine one or more actions being performed during the surgical procedure. For example, vessel sealing, clipping, or any other manipulations of the surgical instruments can be detected based at least in part on the instrument data using machine learning.

The machine learning model can take into consideration one or more temporal inputs, such as sensor information, acoustic information, along with spatial annotations 308 associated with images 302 when detecting features in the surgical data 300. A set of such temporally synchronized inputs from the surgical data 300 that are analyzed together by the machine learning model can be referred to as an “input window” 320. The machine learning model, during inference, operates on the input window 320 to detect a surgical phase represented by the images 302 in the input window 320 (block 202). Each image 302 in the input window 320 is associated with synchronized temporal and spatial annotations, such as measurements at a particular timepoint, including sensor information, acoustic information, and other information. The images 302 that are used by the machine learning models may or may not be sequential.

The input from the surgical instrument system 362 and the audio/video system 364 is temporally synchronized, in one or more examples. A set of such temporally synchronized inputs from the surgical data 300 that are analyzed together by the machine learning model can be referred to as an “input window” 320. The machine learning model, during inference, operates on the input window 320 to detect a surgical phase represented by the images in the input window 320 (block 202). Each input window 320 can include multiple data streams from different sources: one or more images 302 (or video), synchronized temporal and spatial data, such as measurements including sensor measurements, acoustic information, and other information that is used by the machine learning model(s) to detect/detect one or more aspects autonomously.

Aspects also include temporally synchronizing the video data from the surgical instrument system 362 and audio/video system 364. The synchronization includes identifying image(s) 302 from the video data associated with a manipulation of a surgical instrument at a timepoint t1. Alternatively, the synchronization includes identifying the surgical instrumentation data associated with an image 302 at a timepoint t2. In one or more examples, the surgical instrument system 362 and the audio/video system 364 operate using synchronized clocks, and include timestamps from such clocks when the respective data is recorded. The timestamps from the synchronized clocks can be used to synchronize the two data streams. Alternatively, the surgical instrument system 362 and the audio/video system 364 operate on a single clock, and the timestamps can be used to synchronize the respective data streams.

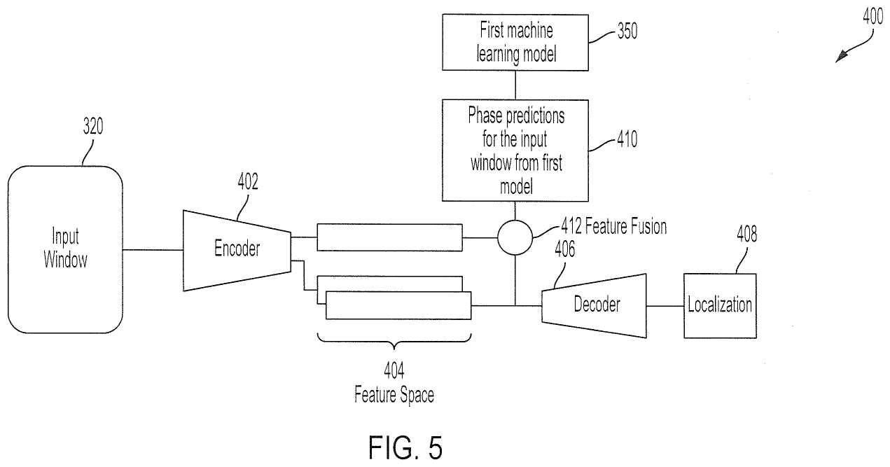

Further, the method 200 of FIG. 3 includes training and using (inference phase) a second machine learning model to detect structural data in the surgical data in the input window 320, at block 204. FIG. 5 depicts a second machine learning model 400 used to detect structures in the surgical data according to one or more aspects. The second machine learning model 400 can be a computer vision model. The second machine learning model 400 can be a combination of one or more artificial neural networks, such as encoders, Recurrent Neural Networks (RNN, e.g., LSTM, GRU, etc.), CNNs, Temporal Convolutional Neural Networks (TCNs), decoders, Transformers, other deep neural networks, etc. In some aspects, the second machine learning model 400 uses an architecture such as DeepLabv3, PSPNet, or any other architecture. In some aspects, the second machine learning model 400 includes an encoder 402 that is trained using weak labels (such as lines, ellipses, local heatmaps, or rectangles) or full labels (segmentation masks, heatmaps) to detect features in the surgical data. In some cases, full labels can be automatically generated from weak labels by using trained machine learning models). In some other cases, the full labels can be transformed into weak labels (e.g., segmentation masks to heatmaps). This transformation of labels can be referred to as “label relaxation,” which allows the machine learning model to learn different parts of the structure with different weights/importance. The encoder or backbone 402 can be implemented using architectures such as ResNet, VGG, or other such neural network architectures. During training, the encoder 402 is trained using input windows 320 that includes images 302 that are annotated with the labels (weak or full).

The encoder 402 generates a feature space 404 from the input window 320. The feature space 404 includes the extracted features from the input window by the encoder 402. The features include one or more labels assigned by the encoder 402 to one or more portions of the surgical data in the input window 320.

The second machine learning model 400 further includes a decoder 406 that detects and outputs localization 408 based on the feature space 404. The localization 408 provides locations, e.g., coordinates, heatmaps, bounding boxes, boundaries, masks, etc., of one or more structures detected in the input window 320. The localization 408 can be specified as multiple sets of coordinates (e.g., polygon), a single set of coordinates (e.g., centroid), or any other such manner without limiting the technical features described herein.

The structures that are detected can include anatomical structures, surgical instruments, and other such features in the input window 320. Anatomical structures that are detected can include organs, arteries, implants, surgical artifacts (e.g., staples, stitches, etc.), etc. Further yet, based on the type of surgical procedure being performed, one or more of the detected anatomical structures can be identified as critical structures for the success of the procedure. The surgical instruments that are detected can include clamps, staplers, knives, scalpels, sealer, divider, dissector, tissue fusion instrument, etc.

The localization 408, in one or more aspects, is limited to the spatial domain (e.g., bounding box, heatmap, segmentation mask) of the structures detected but uses temporal annotations 306 to enhance temporal consistency of the detections. The temporal annotations 306 can be based on sensor measurements, acoustic information, and other such data that is captured at the time of capturing the respective images 302.

In one or more aspects, the decoder 406 further uses information output by the first machine learning model 350, including the phase data. The phase information is injected as a prior during training the second machine learning model 400. The temporal information that is provided by the phase information is used to refine confidence of the detection of structural data in one or more aspects. In one or more aspects, the temporal information is fused (412) with the feature space 404, and the resulting fused information is used by the decoder 406 to output the localization 408 of the structural data.

The feature fusion 412 can be based on transform-domain image fusion algorithms to implement an image fusion neural network (IFNN). For example, an initial number of layers in the IFNN extract salient features from the temporal information output by the first model and the feature space 404. Further, the extracted features are fused by an appropriate fusion rule (e.g., elementwise-max, elementwise-min, elementwise-mean, etc.) or a more complex learning-based neural network module designed to learn to weight and fuse input data (e.g., using attention modules). The fused features are reconstructed by subsequent layers of the IFNN to produce input data, such as an informative fusion image, for the decoder 406 to analyze. Other techniques for fusing the features can be used in other aspects.

The localization 408 can further include a measure of the uncertainty of the processing, i.e., how confident the second machine learning model 400 is that the data points resulting from the processing are correct. The measure represents a confidence score of the second machine learning model's outputs. The confidence score is a measure of the reliability of the detection from the second machine learning model 400. For example, a confidence score of 95 percent or 0.95 means that there is a probability of at least 95 percent that the detection is reliable. The confidence score can be computed as a distance transform from the central axis of structure (i.e., how close from the centroid of the structure) to attenuate detections near the boundaries. The confidence score can also be computed as a probabilistic formulation of the second machine learning model 400 (e.g., Bayesian deep learning, probabilistic outputs like SoftMax or sigmoid functions, etc.). In some aspects, the confidence scores for various detections are scaled and/or normalized within a certain range, e.g., [0, 1].

In some aspects, the second machine learning model 400 uses a first setting to detect the structures in the input window 320. The first setting includes a set of particular values to be assigned to hyperparameters of the second machine learning model 400, for example. The first setting is based on the training of the second machine learning model 400 to detect structural data in an input.

Referring to the flowchart in FIG. 3, the method 200 further includes distinguishing specific anatomical structures from the structural data that is identified by the second machine learning model 400, at block 206. In one or more aspects, the second machine learning model 400 is reused with updated (second) settings to identify particular anatomical structures from the structures that are detected (in step 204). Alternatively, another machine learning model (a third machine learning model) is trained and used to identify the particular anatomical structures from detected structural data. The third machine learning model can have the same structure as the second machine learning model 400 and use feature fusion 412 to take advantage of the phase information detected by the first machine learning model 350, and the structures detected by the second machine learning model 400. The second setting used for identifying the anatomical structures includes a set of particular values to be assigned to hyperparameters, for example. The second setting is based on the training of the third (or second 400) machine learning model to identify particular anatomical structures in an input. Alternatively, the third machine learning model is trained to categorize the one or more structures that are detected by the second machine learning model. Such categorization can include identifying different types of anatomical structures and surgical instruments. The categorization can be performed on the one or more localizations 408, e.g., bounding boxes, output by the second machine learning model 400.

The localization output by the third machine learning model provides identification of specific anatomical structures in the images 302, for example, cystic artery 12 and cystic duct 14. The localization can be represented as coordinates in the images 302 that map to pixels depicting the identified anatomical structures in the images 302. In some aspects, output of the third machine learning model is an augmented heatmap, a segmentation mask, a point cloud or other type of landmarks for each of the anatomical structures. These landmarks can be used to generate augmented videos, as input to further machine learning models for trajectory estimation or tracking, to provide statistics about positions of anatomy and tools and use it for real-time or post-operative analytics, etc.

In one or more aspects, the third machine learning model facilitates identifying an anatomical structure even when it is occluded by at least one other structure in the input window 320. The occlusion can be overcome by using spatio-temporal information of the anatomical structure from other input windows 320. For example, spatio-temporal windows are used when training the machine learning model, to facilitate learning motion dynamics across time and improve segmentation and re-identification of structures.

In an example aspect, for laparoscopic cholecystectomy procedures, the second machine learning model 400 is trained under two different settings: a single combined critical structures class, and separate cystic artery and cystic duct classes. The trained second machine learning model 400 is used separately, once for detection of structures, and again for distinguishing the detected structures. In an example, a training dataset containing 100,000 frames from 1000 videos, labeled under expert guidance, is used for training the second machine learning model 400. It is understood that a different training dataset with different number of elements can be used to train the second (or any other) machine learning model 400 in other implementations. In the above-described example, the second machine learning model 400 detects the presence of structures with a confidence score of 95% with the settings from combined structure classes, and 91% when distinguishing/identifying cystic artery 12 and cystic duct 14 anatomical structures from the detected structures. This is comparable with agreement between human annotators (88% before feedback, 92% after feedback). Accordingly, aspects described herein provide a technical solution to detect anatomical structures, and further analyze the detected anatomical structures to distinguish between specific types of anatomical structures. The aspects integrate the technical solutions into a practical application to use machine learning model(s) to use computer vision to perform the analysis on an input video. In one or more aspects, the analysis is performed substantially in real-time, as the video is being streamed, and during a surgical procedure being performed. The output of the analysis facilitates providing feedback to the medical personnel, for example, by adding a visual overlay on the video stream. Accordingly, a practical solution is provided. Further, an improvement to computer-assisted surgical system (e.g., laparoscopic surgical system, or any other surgical system which provides a live endoscopic video) is provided by providing such feedback, which can enhance the quality of the surgical procedure being performed.

The method 200 of FIG. 3 further includes generating an augmented visualization of the surgical view using the data points obtained from the processing, at block 208. The augmented visualization can include, for example, displaying segmentation masks or probability maps over identified anatomical structures, or specific points of interest in the surgical data 300.

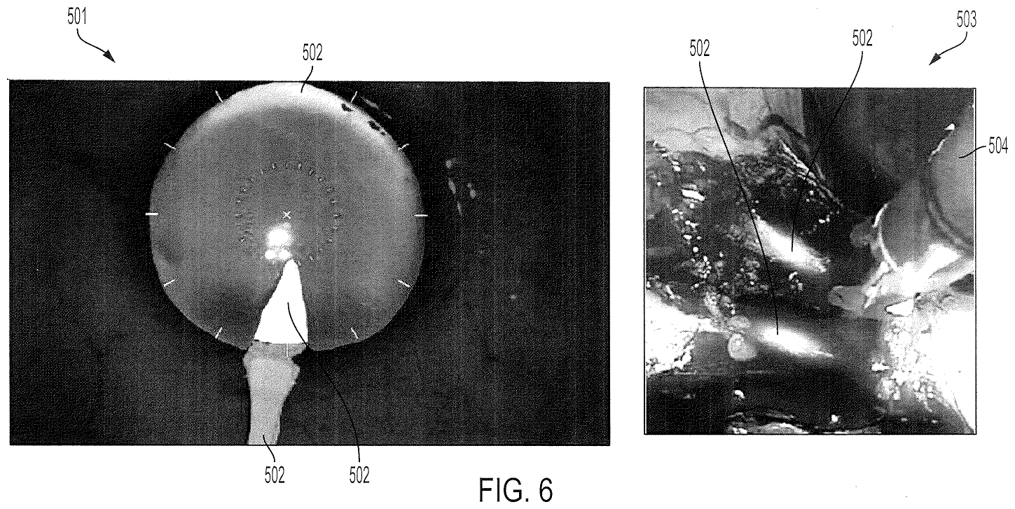

FIG. 6 depicts example augmented visualizations of surgical views generated according to one or more aspects. It is understood that those shown are examples and that various other augmented visualizations can be generated in other aspects. Images captured during an eye surgery are depicted in the augmented visualizations 501, 503. The augmented visualizations 501, 503 are from different phases in the surgical procedure, and accordingly, the anatomical structures, surgical instruments, and other details in the surgical view are different. Further, according to the phase of the surgical procedure, the critical anatomical structures that are identified also change. In the augmented visualization 501, the iris and a specific portion of the iris that is to be operated on are the identified anatomical structures using a graphical overlay 502. The sclera, which is also seen, is not marked, for example, because it may not be deemed as a “critical structure” for the surgical procedure or surgical phase being performed. In the augmented visualization 501, the internals of the eye are seen, such as ciliary muscle, vitreous gel, fovea, choroid, macula, retina, etc. The augmented visualization 503 depicts a snapshot from a laparoscopic cholecystectomy. Among the anatomical structures that are seen, the critical anatomical structures that are to be operated upon, or which are requested to be identified by a user, are marked using graphical overlays 502.

A user can configure which detections from the machine learning system 100 are to be displayed by the augmentor 175. For example, the user can configure to display overlays 502 on a partial set of the detections, with the other detections not being marked in the augmented reality device 180. Further, the user can configure one or more thresholds that determine when to generate an alert based on one or more metrics (e.g., certainty, accuracy, etc.) associated with the detections. The user can further configure the attributes to be used to generate the user feedback, such as the overlays 502. For example, the color, the border, the transparency, the priority, the audible sound, and other such attributes of the user feedback can be configured.

“Critical anatomical structures” can be specific to the type of surgical procedure being performed and identified automatically. Additionally, the surgeon or any other user can configure the system 100 to identify particular anatomical structures as critical for a particular patient. The selected anatomical structures are critical to the success of the surgical procedure, such as anatomical landmarks (e.g., Calot triangle, Angle of His, cystic artery 12, cystic duct 14, etc.) that need to be identified during the procedure or those resulting from a previous surgical task or procedure (e.g., stapled or sutured tissue, clips, etc.).

Further, the augmented visualizations 501, 503 can mark surgical instruments in the surgical data 300 using graphical overlays 502. The surgical instruments are identified by the machine learning models, as described herein. In one or more aspects, a surgical instrument is only marked if it is within a predetermined threshold proximity of an anatomical structure. In some aspects, the surgical instrument is only marked if it is within a predetermined threshold proximity of a critical anatomical structure. In some aspects, a surgical instrument is always marked with a graphical overlay 502, but the opacity (or any other attribute) of the graphical overlay 502 is varied based on an importance-score associated with the surgical instrument. The importance-score can be based on the surgical procedure being performed. For example, during a knee-arthroscopy for a meniscus injury, an arthroscopic scissor, a suture cutter, a meniscus retractor, or other such surgical instruments may have a larger importance-score compared to an arthroscopic punch, a biter, etc. The importance-scores for the surgical instruments can be configured by the user, and can be set by default based on the type of the surgical procedure being performed. The graphical overlays 502 for other detected features, such as anatomical structures, are also adjusted in the same manner as those for a surgical instrument.

Here, “marking” an anatomical structure, surgical instrument, or other features in the surgical data includes visually highlighting that feature for the surgeon or any other user by using a graphical overlay 502. The graphical overlay 502 can include a heatmap, a contour, a bounding box, a mask, a highlight, or any other such visualization that is overlaid on the images 302 from the surgical data 300 that are being displayed to the user. Further, in one or more aspects, the specific anatomical structures that are identified are marked using predetermined values that are assigned to respective anatomical structures. For example, as shown in FIG. 1, the cystic artery 12 is marked using a first color value (e.g., purple), and the cystic duct 14 is marked using a second color value (e.g., green). It can be appreciated that visual attributes other than color or a combination thereof can also be assigned to specific anatomical structures. The assignment of the visual attributes to respective anatomical structures can be user configurable. The examples herein depict using masks and heatmaps as the graphical overlays 502. However, different techniques can be used in other aspects.

Various visual attributes of the graphical overlay 502, such as colors, transparency, visual-pattern, line thickness, etc., can be adjusted. In addition, the graphic overlay 502 can include annotations. The annotation can identify the anatomical structure(s), objects that are marked using the graphic overlay 502 based on the detection by the second machine learning model 400. Additionally, the annotation can include a note, a sensor measurement, or other such information for the user.

In one or more aspects, a user can adjust the attributes of the graphic overlays 502. For example, the user can select a type of highlighting, a color, a line thickness, a transparency, a shading pattern, a label, an outline, or any other such attributes to be used to generate and display the graphical overlay on the images 302. In some aspects, the color and/or transparency of the graphical overlay 502 is modulated based on the confidence score associated with the identification of the underlying anatomical structure or surgical instrument by the machine learning model(s).

In some aspects, the graphical overlays 502 are used to provide a critical structure warning. Referring to the flowchart, the method 200 includes predicting whether the surgeon is operating within a predetermined proximity of a critical anatomical structure, at block 210. Such a determination can be made based on a surgical instrument being within a predetermined proximity (e.g., 0.5 millimeters, 0.2 millimeters, etc.) of a critical anatomical structure. If a surgical instrument is determined to be within the predetermined threshold of a critical anatomical structure, one or more preventive measures are taken, at block 212.

The preventive measures can include generating and displaying a graphical overlay 502 on the surgical view to indicate a user feedback (e.g., warning/alert, annotation, notification, etc.). Alternatively, or in addition, preventive measures can be integrated into a robotic workflow in response to the estimates made by the machine learning models described herein. For example, operating parameters of one or more surgical instruments are adjusted (e.g., limited/restrained) to prevent injury to the patient. For example, during a ureteroscopy, to prevent injury to ureters and/or pulmonary vasculature, the energy level of monopolar instruments is reduced when dissecting in the proximity of neurovascular bundles. Additional preventive measures can also be taken in other aspects by adjusting operating parameters, such as, speed, rotations, vibrations, energy, etc., that can facilitate prohibiting (or enhancing) one or more actions being performed using the surgical instrument.

Aspects of the technical solutions described herein improve surgical procedures by improving the safety of the procedures. Further, the technical solutions described herein facilitate improvements to computing technology, particularly computing techniques used during a surgical procedure. Aspects of the technical solutions described herein facilitate one or more machine learning models, such as computer vision models, to process images obtained from a live video feed of the surgical procedure in real-time using spatio-temporal information. The machine learning models use techniques such as neural networks to use information from the live video feed and (if available) robotic sensor platform to detect and distinguish one or more features, such as anatomical structures, surgical instruments, in an input window of the live video feed, and further refine the predictions using additional machine learning models that can predict a phase of the surgical procedure. The additional machine learning models are trained to identify the surgical phase(s) of the procedure and instruments in the field of view by learning from raw image data and instrument markers (bounding boxes, lines, key points, etc.). When in a robotic procedure, the computer vision models can also accept sensor information (e.g., instruments enabled, mounted, etc.) to improve the predictions. Computer Vision models that predict instruments and critical anatomical structures use temporal information from the phase prediction models to improve the confidence of the predictions in real-time. It should be noted that an output of a machine learning model can be generally referred to as “prediction” unless specified otherwise.

The predictions and the corresponding confidence scores are used to generate and display graphical overlays to the surgeon and/or other users in an augmented visualization of the surgical view. The graphical overlays can mark critical anatomical structures, surgical instruments, surgical staples, scar tissue, results of previous surgical actions, etc. The graphical overlays can further show a relationship between the surgical instrument(s) and one or more anatomical structures in the surgical view and thus, guide the surgeon and other users during the surgery. The graphical overlays are adjusted according to the user's preferences and/or according to the confidence scores of the predictions.

By using machine learning models, and computing technology to predict and mark various features in the surgical view, in real-time, aspects of the technical solutions facilitate the surgeons to replace visualizations based on external contrast agents (e.g., Indocyanine green (ICG), Ethiodol, etc.) that have to be injected into the patient. Such contrast agents may not always be available to use because of the patient's preconditions or other factors. Accordingly, aspects of the technical solutions described herein provide a practical application in surgical procedures. In some aspects, the contrast agents can be used in addition to the technical solutions described herein. The operator, for example, the surgeon, can switch on/off either (or both) visualizations, the contrast agent based, or the graphical overlays 502.

Further yet, aspects of the technical solutions described herein address technical challenges of predicting complex features in a live video feed of a surgical view in real-time. The technical challenges are addressed by using a combination of various machine learning techniques to analyze multiple images in the video feed. Additionally, technical challenges exist to determine relative depth in the images when determining if a surgical instrument is within a predetermined proximity of a critical anatomical structure. Aspects of the technical solutions described herein provide machine learning techniques that facilitate training a depth estimation algorithm to retrieve a proxy for relative depth in the images. Further yet, to address the technical challenge of real-time analysis and augmented visualization of the surgical view, aspects of the technical solutions described herein predict the present state of the surgical view at a constant frame rate and update the present state using the machine learning models at a predetermined frame rate.

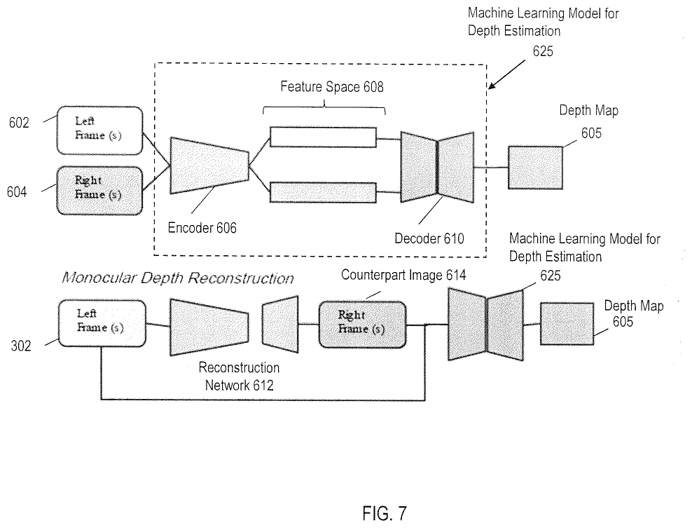

FIG. 7 depicts flow diagrams of depth estimation to retrieve a proxy for relative depth in the image. The surgical procedure is being performed in 3D space, and hence, determining the proximity of surgical instruments and anatomical structures has to be performed in 3D space. However, the images 302 representing the surgical view are typically 2D. Therefore, a depth map of the surgical view has to be estimated based on the 2D images 302. Depth map calculation is a technical challenge in computing technology, as the calculation is expensive both in computing resources and in time. The aspects described herein address the technical challenge by using artificial neural network architecture that improves the runtime of calculating the depth map 605 in real-time. Further, the surgical view may be captured using a monocular image capture device (e.g., a single camera), which can adversely affect the estimation of the depth map. It should be noted that “depth map” can represent a disparity map, a stereo map, a distance map, or any other such data structures.

Aspects described herein address such technical challenges and provide a depth map for the surgical view in real-time.

FIG. 7 depicts training a machine learning model 625 used to estimate a depth map 605 of features seen in the surgical view. The machine learning model 625 is trained using a pair of stereo frames that are captured using a stereo image capture device (not shown). The stereo image capture device captures two frames, referred to herein as a left frame 602 and a right frame 604. It is understood that in other aspects, the stereo image capture can produce a top frame and a bottom frame or any other pair of images that capture a scene in the field of view of the stereo image capture device. The machine learning model 625 can also be trained to extract the depth map 605 using simulated data, for which exact depth is known and left/right projections can be taken. Additionally, models can also be trained with spatio-temporal information (e.g. using a window of frames/sensors and other inputs, like other models described herein).

The machine learning model is based on artificial neural network architecture. The neural network architecture includes an encoder 606 that is trained to extract features from the left frame 602 and the right frame 604, respectively. The features that are extracted into a feature space 608 can be based on filters, such as a Sobel filter, Prewitt operator, or other feature detection operators such as convolutional operators. Further, a decoder 610 determines the depth map 605 by matching the extracted features from the left frame 602 and the right frame 604 and computing the coordinates for each point in the scene based on the matched features. The encoder 606 and the decoder 610 each include RNN, CNN, Transformer, or other such neural networks. The depth map 605 provides the depth of each pixel in the scene captured by the stereo pair. During training, the ground truth of the depth map 605 is known, and accordingly, the encoder 606 and the decoder 610 are trained to find accurately matching features, and the depth of each pixel in the depth map 605 based on the matching features. The depth map 605 is an image with the same dimensions as the left frame 602 and the right frame 604, with the value of each pixel in the depth map 605 representing the depth of each of the points captured in the stereo pair.

During runtime, because a stereo image capture device can be absent, a monocular depth reconstruction is performed using the trained machine learning model 625. The images 302 that are captured are used to reconstruct corresponding counterpart images 614 using a reconstruction network (RecNet) 612. The original images 302 and the corresponding counterpart images 614 from the reconstruction network 612 are used as a stereo pair of images (left and right) that is input to the trained machine learning model 625 for estimating the depth map 605.

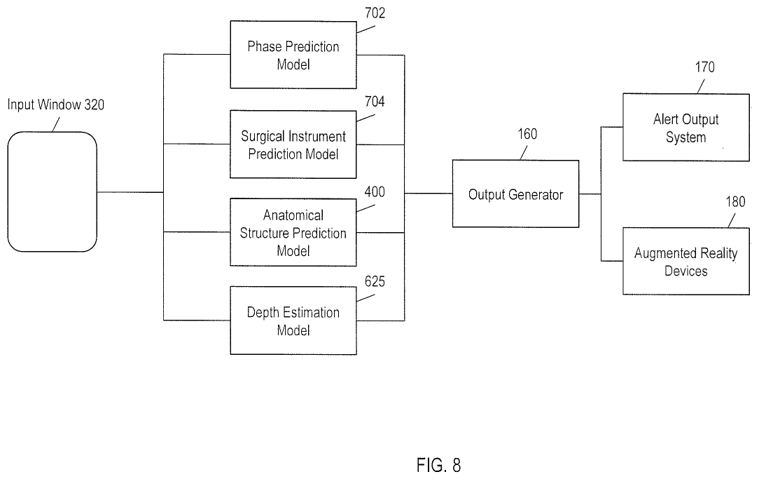

FIG. 8 depicts a flow diagram of automatic prediction of anatomical structures in surgical data using machine learning according to one or more aspects. The input window 320 is input to the model execution system 140 of FIG. 2 that uses a phase prediction model 702, which is a machine learning model, to predict the phase of the surgical procedure being performed. Further, the input window 320 is analyzed by the second machine learning model 400 to predict one or more anatomical structures. Further yet, surgical instruments in the surgical data are predicted using a surgical instrument prediction model 704, which is another machine learning model. The surgical instrument prediction model 704 can be substantially similar in architecture to the second machine learning model 400, which is used for anatomical structure prediction. Further yet, the input window is analyzed by the depth estimation model 625 to generate the depth map 605. The machine learning models are trained using training data, which is substantially similar in structure to the surgical data 300. It should be noted that although separate machine learning models are described herein for detecting separate features of the surgical data, it should be understood that in some aspects, a single machine learning model, or a different combination of machine learning models (e.g., two models, three models) can be used to detect the features. The surgical training data can be recorded surgical data from prior surgical procedures or simulated surgical data, as described herein. The training data is pre-processed, for example, manually, to know the ground truth, and adjust the hyperparameters, and other parameters associated with the machine learning models during the training phase. The machine learning models are deemed to be trained once the output predictions from the models are within a predetermined error threshold of the ground truth and the corresponding confidence scores of the predictions are above a predetermined threshold.

During an inference phase, the trained machine learning models are input live surgical data 300, that has not been pre-processed. The machine learning models, in the inference phase, generate the predictions. One or more machine learning models also output corresponding confidence scores associated with the predictions.

The outputs from each of the machine learning models are used by the output generator 160 to provide augmented visualization via the augmented reality devices 180. The augmented visualization can include the graphical overlays 502 being overlaid on the corresponding features (anatomical structure, surgical instrument, etc.) in the image(s) 302.

The output generator 160 can also provide a user feedback via the alert output system 170 in some aspects. The user feedback can include highlighting using graphical overlays 502 one or more portions of the image(s) 302 to depict proximity between the surgical instrument(s) and anatomical structure(s). Alternatively, or in addition, the user feedback can be displayed in any other manner, such as a message, an icon, etc., being overlaid on the image(s) 302.

In some aspects, to facilitate a real-time performance, the input window 320 is analyzed at a predetermined frequency, such as 5 times per second, 3 times per second, 10 times per second, etc. The analysis results in identification of locations of anatomical structures and surgical instruments in the images 302 that are in the input window 320. It can be appreciated that the video of the surgical procedure includes images 302 that are between two successive input windows 320. For example, if the video is captured at 60 frames per second, and if the input window 320 includes 5 frames, and if the input window 320 is analyzed 5 times per second, then a total of 25 frames from the captured 60 are analyzed. The remaining 35 frames are in between two successive input windows 320. It is understood that the capture speed, input window frequency, and other parameters can vary from one aspect to another, and that above numbers are examples.

For the frames, i.e., images 302, between two successive input windows 320, the locations of the anatomical structures and surgical instruments are predicted based on the locations predicted in the most recent input window 320. For example, a movement vector of the surgical instrument can be computed based on the changes in the location of the surgical instrument in the frames in the prior input window 320. The movement vector can be computed using a machine learning model, such as a deep neural network. The movement vector is used to predict the location of the surgical instrument in the subsequent frames after the input window 320, until a next input window 320 is analyzed.

The location of the anatomical structure(s) predicted by the machine learning model is also predicted in the frames between two successive input windows 320 in the same manner. The graphical overlays 502 that are used to overlay the images 302 to represent the predicted features (surgical instruments, anatomical structures, etc.) are accordingly adjusted, if required, based on the predicted locations. Accordingly, a smooth visualization, in real time, is provided to the user with lesser computing resources being used. In some aspects, the graphical overlays 502 can be configured to be switched off by the user, for example, the surgeon, and the system works without overlays 502, rather only generating the overlays 502 and/or other types of user feedback when an alert is to be provided (e.g., instrument within predetermined vicinity of an anatomical structure).

Complications such as bile duct injury during a surgery like laparoscopic cholecystectomy can seriously injure a patient. Technical solutions are provided to facilitate computer assistance during the surgery to prevent complications by detecting and highlighting critical anatomical structures, such as the cystic duct and cystic artery, using machine learning. According to some aspects, a computer vision system is trained to detect several structures in a live video of the surgery, and further to distinguish between the structures, such as the artery and the duct, despite their similar appearance.

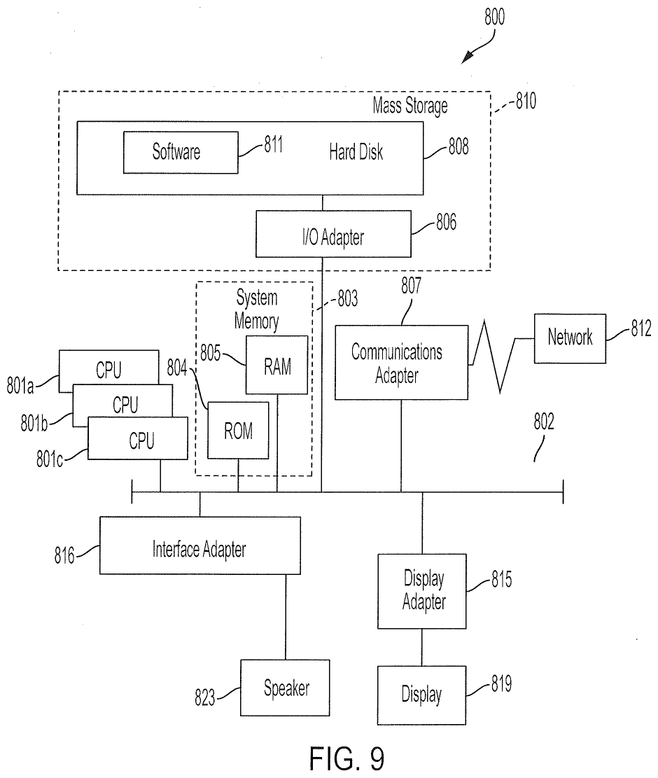

Turning now to FIG. 9, a computer system 800 is generally shown in accordance with an aspect. The computer system 800 can be an electronic, computer framework comprising and/or employing any number and combination of computing devices and networks utilizing various communication technologies, as described herein. The computer system 800 can be easily scalable, extensible, and modular, with the ability to change to different services or reconfigure some features independently of others. The computer system 800 may be, for example, a server, desktop computer, laptop computer, tablet computer, or smartphone. In some examples, computer system 800 may be a cloud computing node. Computer system 800 may be described in the general context of computer system executable instructions, such as program modules, being executed by a computer system. Generally, program modules may include routines, programs, objects, components, logic, data structures, and so on that perform particular tasks or implement particular abstract data types. Computer system 800 may be practiced in distributed cloud computing environments where tasks are performed by remote processing devices that are linked through a communications network. In a distributed cloud computing environment, program modules may be located in both local and remote computer system storage media, including memory storage devices.

As shown in FIG. 9, the computer system 800 has one or more central processing units (CPU(s)) 801a, 801b, 801c, etc. (collectively or generically referred to as processor(s) 801). The processors 801 can be a single-core processor, multi-core processor, computing cluster, or any number of other configurations. The processors 801, also referred to as processing circuits, are coupled via a system bus 802 to a system memory 803 and various other components. The system memory 803 can include one or more memory devices, such as a read-only memory (ROM) 804 and a random access memory (RAM) 805. The ROM 804 is coupled to the system bus 802 and may include a basic input/output system (BIOS), which controls certain basic functions of the computer system 800. The RAM is read-write memory coupled to the system bus 802 for use by the processors 801. The system memory 803 provides temporary memory space for operations of said instructions during operation. The system memory 803 can include random access memory (RAM), read-only memory, flash memory, or any other suitable memory systems.

The computer system 800 comprises an input/output (I/O) adapter 806 and a communications adapter 807 coupled to the system bus 802. The I/O adapter 806 may be a small computer system interface (SCSI) adapter that communicates with a hard disk 808 and/or any other similar component. The I/O adapter 806 and the hard disk 808 are collectively referred to herein as a mass storage 810.

Software 811 for execution on the computer system 800 may be stored in the mass storage 810. The mass storage 810 is an example of a tangible storage medium readable by the processors 801, where the software 811 is stored as instructions for execution by the processors 801 to cause the computer system 800 to operate, such as is described hereinbelow with respect to the various Figures. Examples of computer program product and the execution of such instruction is discussed herein in more detail. The communications adapter 807 interconnects the system bus 802 with a network 812, which may be an outside network, enabling the computer system 800 to communicate with other such systems. In one aspect, a portion of the system memory 803 and the mass storage 810 collectively store an operating system, which may be any appropriate operating system to coordinate the functions of the various components shown in FIG. 9.

Additional input/output devices are shown as connected to the system bus 802 via a display adapter 815 and an interface adapter 816 and. In one aspect, the adapters 806, 807, 815, and 816 may be connected to one or more I/O buses that are connected to the system bus 802 via an intermediate bus bridge (not shown). A display 819 (e.g., a screen or a display monitor) is connected to the system bus 802 by a display adapter 815, which may include a graphics controller to improve the performance of graphics-intensive applications and a video controller. A keyboard, a mouse, a touchscreen, one or more buttons, a speaker, etc., can be interconnected to the system bus 802 via the interface adapter 816, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 9, the computer system 800 includes processing capability in the form of the processors 801, and, storage capability including the system memory 803 and the mass storage 810, input means such as the buttons, touchscreen, and output capability including the speaker 823 and the display 819.

In some aspects, the communications adapter 807 can transmit data using any suitable interface or protocol, such as the internet small computer system interface, among others. The network 812 may be a cellular network, a radio network, a wide area network (WAN), a local area network (LAN), or the Internet, among others. An external computing device may connect to the computer system 800 through the network 812. In some examples, an external computing device may be an external web server or a cloud computing node.

It is to be understood that the block diagram of FIG. 9 is not intended to indicate that the computer system 800 is to include all of the components shown in FIG. 9. Rather, the computer system 800 can include any appropriate fewer or additional components not illustrated in FIG. 9 (e.g., additional memory components, embedded controllers, modules, additional network interfaces, etc.). Further, the aspects described herein with respect to computer system 800 may be implemented with any appropriate logic, wherein the logic, as referred to herein, can include any suitable hardware (e.g., a processor, an embedded controller, or an application-specific integrated circuit, among others), software (e.g., an application, among others), firmware, or any suitable combination of hardware, software, and firmware, in various aspects.

The reports/views/annotations and other information described herein is added to an electronic medical record (EMR) in one or more cases. In some aspects, the information about specific surgical procedures can be stored in the patient record associated with the patient that was operated upon during the surgical procedure. Alternatively, or in addition, the information is stored in a separate database for later retrieval. The retrieval can be associated with the patient's unique identification, such as EMR-identification, social security number, or any other unique identifier. The stored data can be used to generate patient-specific reports. In some aspects, information can also be retrieved from the EMR to enhance one or more operations described herein. In one or more aspects, an operational note may be generated, which includes one or more outputs from the machine learning models. The operational note may be stored as part of the EMR.

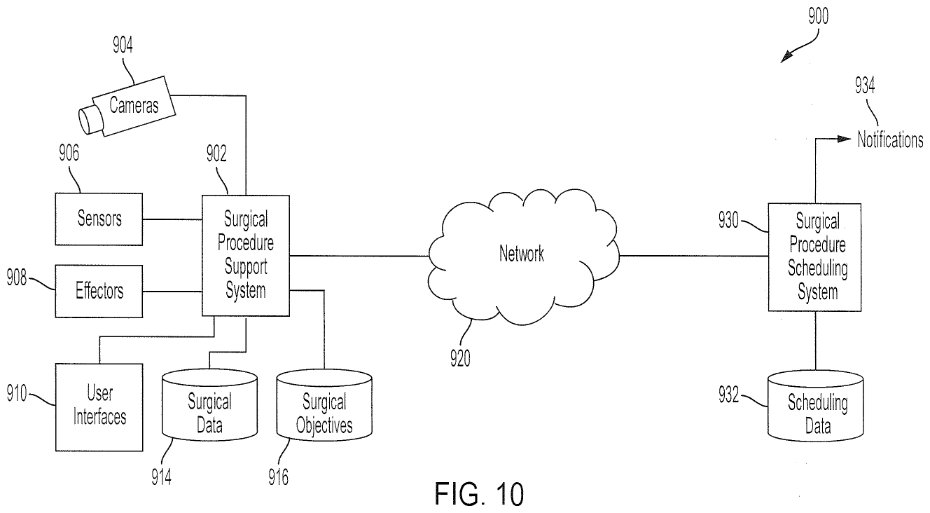

FIG. 10 depicts a surgical procedure system 900 in accordance with one or more aspects. The example of FIG. 10 depicts a surgical procedure support system 902 configured to communicate with a surgical procedure scheduling system 930 through a network 920. The surgical procedure support system 902 can include or may be coupled to the system 100 of FIG. 2. The surgical procedure support system 902 can acquire image data, such as images 302 of FIG. 4, using one or more cameras 904. The surgical procedure support system 902 can also interface with a plurality of sensors 906 and effectors 908. The sensors 906 may be associated with surgical support equipment and/or patient monitoring. The effectors 908 can be robotic components or other equipment controllable through the surgical procedure support system 902. The surgical procedure support system 902 can also interact with one or more user interfaces 910, such as various input and/or output devices. The surgical procedure support system 902 can store, access, and/or update surgical data 914 associated with a training dataset and/or live data as a surgical procedure is being performed. The surgical procedure support system 902 can store, access, and/or update surgical objectives 916 to assist in training and guidance for one or more surgical procedures.

The surgical procedure scheduling system 930 can access and/or modify scheduling data 932 used to track planned surgical procedures. The scheduling data 932 can be used to schedule physical resources and/or human resources to perform planned surgical procedures. Based on the surgical maneuver as predicted by the one or more machine learning models and a current operational time, the surgical procedure support system 902 can estimate an expected time for the end of the surgical procedure. This can be based on previously observed similarly complex cases with records in the surgical data 914. A change in a predicted end of the surgical procedure can be used to inform the surgical procedure scheduling system 930 to prepare the next patient, which may be identified in a record of the scheduling data 932. The surgical procedure support system 902 can send an alert to the surgical procedure scheduling system 930 that triggers a scheduling update associated with a later surgical procedure. The change in schedule can be captured in the scheduling data 932. Predicting an end time of the surgical procedure can increase efficiency in operating rooms that run parallel sessions, as resources can be distributed between the operating rooms. Requests to be in an operating room can be transmitted as one or more notifications 934 based on the scheduling data 932 and the predicted surgical maneuver.

As surgical maneuvers and steps are completed, progress can be tracked in the surgical data 914, and status can be displayed through the user interfaces 910. Status information may also be reported to other systems through the notifications 934 as surgical maneuvers are completed or if any issues are observed, such as complications.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network, and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source-code or object code written in any combination of one or more programming languages, including an object-oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some aspects, electronic circuitry including, for example, programmable logic circuitry, field-programmable gate arrays (FPGA), or programmable logic arrays (PLA) may execute the computer-readable program instruction by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to aspects of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other devices to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various aspects of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects of the present invention have been presented for purposes of illustration but are not intended to be exhaustive or limited to the aspects disclosed. Many modifications and variations will be apparent to those of ordinary skill in the art without departing from the scope and spirit of the described aspects. The terminology used herein was chosen to best explain the principles of the aspects, the practical application or technical improvement over technologies found in the marketplace, or to enable others of ordinary skill in the art to understand the aspects described herein.

Various aspects of the invention are described herein with reference to the related drawings. Alternative aspects of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains,” or “containing,” or any other variation thereof are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any aspect or design described herein as “exemplary” is not necessarily to be construed as preferred or advantageous over other aspects or designs. The terms “at least one” and “one or more” may be understood to include any integer number greater than or equal to one, i.e., one, two, three, four, etc. The terms “a plurality” may be understood to include any integer number greater than or equal to two, i.e., two, three, four, five, etc. The term “connection” may include both an indirect “connection” and a direct “connection.”

The terms “about,” “substantially,” “approximately,” and variations thereof are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A computer-implemented method comprising:
detecting, using a first configuration of a neural network, a plurality of structures in a video of a laparoscopic surgical procedure;
identifying, using a second configuration of the neural network, from the plurality of structures, a first type of anatomical structure and a second type of anatomical structure; and
generating an augmented video, the generating comprising annotating the video with the first type of anatomical structure and the second type of anatomical structure.

2. The computer-implemented method of claim 1, wherein the surgical procedure is a laparoscopic cholecystectomy, the first type of anatomical structure is a cystic artery, and the second type of anatomical structure is a cystic duct.

3. The computer-implemented method of claim 1, wherein an anatomical structure, from the plurality of structures, occludes at least one other anatomical structure from the plurality of structures in a frame of the video.

4. The computer-implemented method of claim 3, wherein the second configuration includes using one or more temporal models to provide context to the frame.

5. The computer-implemented method of claim 1, wherein the neural network is trained to generate the second configuration based on weak labels.

6. The computer-implemented method of claim 1, wherein the video is a live video stream of the surgical procedure.

7. The computer-implemented method of claim 1, wherein the first type of anatomical structure is annotated differently than the second type of anatomical structure.

8. The computer-implemented method of claim 1, wherein the annotating comprises adding, to the video, at least one from a mask, a bounding box, and a label.

9. A system comprising:
a training system configured to use a training dataset to train one or more machine learning models;
a data collection system configured to capture a video of a surgical procedure being performed;
a machine learning model execution system configured to execute the one or more machine learning models to perform a method comprising:
detecting a plurality of structures in the video by using a first configuration of the one or more machine learning models; and
identifying, from the plurality of structures, at least one type of anatomical structure by using a second configuration of the one or more machine learning models; and
an output generator configured to generate an augmented video by annotating the video to mark the at least one type of anatomical structure.

10. The system of claim 9, wherein a first machine learning model is trained to detect the plurality of structures and a second machine learning model is trained to identify the at least one type of anatomical structure from the plurality of structures.

11. The system of claim 9, wherein a same machine learning model is used to detect the plurality of structures and to identify the at least one type of anatomical structure from the plurality of structures.

12. The system of claim 11, wherein the same machine learning model, to detect the plurality of structures, uses the first configuration, which comprises a first set of hyperparameter values, and to identify the at least one type of anatomical structure, uses the second configuration, which comprises a second set of hyperparameter values.

13. The system of claim 9, wherein the training system is further configured to train a third machine learning model to identify at least one surgical instrument from the plurality of structures.

14. A computer program product comprising a memory device having computer executable instructions stored thereon, which when executed by one or more processors cause the one or more processors to perform a method for prediction of features in surgical data using machine learning, the method comprising:
detecting, using a neural network model, a plurality of structures in an input window comprising one or more images from a video of a surgical procedure, the neural network model is trained using surgical training data;
identifying, using the neural network model, at least one type of anatomical structure in the plurality of structures detected; and
generating a visualization of the surgical procedure by displaying a graphical overlay at a location of the at least one type of anatomical structure in the video of the surgical procedure.

15. The computer program product of claim 14, wherein the neural network model detects the location of the at least one type of anatomical structure based on an identification of a phase of the surgical procedure being performed.

16. The computer program product of claim 14, wherein one or more visual attributes of the graphical overlay are configured based on the at least one type of anatomical structure.

17. The computer program product of claim 16, wherein the one or more visual attributes assigned to the at least one type of anatomical structure are user configurable.

18. The computer program product of claim 14, wherein the neural network model is configured with a first set of hyperparameters to detect the plurality of structures, and with a second set of hyperparameters to identify the at least one type of anatomical structure.

19. The computer program product of claim 14, wherein the neural network model comprises a first neural network for semantic image segmentation and a second neural network for encoding.

20. The computer program product of claim 14, wherein the plurality of structures comprises one or more anatomical structures and one or more surgical instruments.
